(19) United States
(12) Patent Application Publication
(10) Pub. No.: US 2018/0056025 A1
(43) Pub. Date: Mar. 1, 2018

(54) SYSTEM AND METHOD FOR CUSTOMIZED ADDICTION THERAPY BASED ON BIO SIGNAL

(71) Applicant: KOREA INSTITUTE OF SCIENCE AND TECHNOLOGY, Seoul (KR)

(72) Inventors: Wanjoo PARK, Seoul (KR); Laehyun KIM, Seoul (KR); Da-hye KIM, Seoul (KR)

(73) Assignee: KOREA INSTITUTE OF SCIENCE AND TECHNOLOGY, Seoul (KR)

(21) Appl. No.: 15/388,005

(22) Filed: Dec. 22, 2016

(30) Foreign Application Priority Data
Aug. 24, 2016 (KR) 10-2016-0107567

(51) Int. Cl. A61M 21/00; A61B 5/0476; A61B 5/053; A61B 5/08; A61B 5/0488; A61B 5/16

(52) U.S. Cl. CPC A61M 21/00; A61B 5/0476; A61B 5/0533; A61M 2021/005; A61B 5/0488; A61B 5/165; A61B 5/0816

(57) ABSTRACT

Exemplary embodiments relate to a system for customized addiction therapy based on a bio signal, including a sensor configured to measure a bio signal from a user, a state determining unit configured to determine a user state based on the measured bio signal, a cognitive behavioral therapy image determining unit configured to determine a cognitive behavioral therapy image based on the determined user state, and an image providing unit configured to provide the determined cognitive behavioral therapy image to the user, and its method.

### CROSS-REFERENCE TO RELATED APPLICATION

This application claims priority to Korean Patent Application No. 10-2016-0107567, filed on Aug. 24, 2016, and all the benefits accruing therefrom under 35 U.S.C. §119, the contents of which in its entirety are herein incorporated by reference.

### BACKGROUND

### 1. Field

The present disclosure relates to a system and method for addiction therapy, and more particularly, to a system and method for customized addiction therapy using a bio signal.

### [Project Information]

[Project serial number] 1711029657
[Government department name] Ministry of Science, ICT and Future Planning
[Research management specialized organization] National Research Foundation of Korea
[Research program name] Development of original technology for brain science
[Research project name] Development of bio signal-based integrated monitoring and biofeedback technology
[Contribution ratio] 1/1
[Supervision organization] Korea Institute of Science and Technology
[Research period] 2015.10.01-2016.09.30

### 2. Description of the Related Art

Cognitive behavioral therapy is treatment that is used to develop normal social behavioral activities by repeatedly carrying out cognition and behavior modification on the premise that social behavioral dysfunction in human occurs due to false thoughts about events or situations.

Conventional cognitive behavioral therapy is performed by a user according to a fixed scenario set for the therapeutic purpose presented by a medical team. However, this procedure has a practical limitation on continuous transmission of constantly changing states of the user to a clinical or counseling psychologist. Furthermore, there is a limitation on comprehensive state objectification including the level of cognition (attention, execution, etc.), emotion (anger, depression, etc.), and craving of the user based on the received content.

### RELATED LITERATURES

### Patent Literatures

### (Patent Literature 1) Korean Patent No. 10-1308522

### (Patent Literature 2) Korean Patent No. 10-1585830

### (Patent Literature 3) Korean Patent No. 10-0914880

### SUMMARY

To solve the problem faced by a clinical or counseling who has difficulty objectively identifying a user's response to a given virtual environment and situation in cognitive behavioral therapy, the present disclosure is aimed at providing the clinical or counseling psychologist with objective indices by analyzing bio signals of the user. The present disclosure is further aimed at providing a customized cognitive therapy image based on the objective indices.

The problem to be solved by the present disclosure is not limited thereto, and other technical problems not mentioned herein will be clearly understood by those skilled in the art from the following description.

A system for customized addiction therapy based on a bio signal according to an embodiment of the present disclosure includes a sensor configured to measure a bio signal from a user, a state determining unit configured to determine a user state based on the measured bio signal, a cognitive behavioral therapy image determining unit configured to determine a cognitive behavioral therapy image based on the determined user state, and an image providing unit configured to provide the determined cognitive behavioral therapy image to the user.

In an embodiment, the bio signal may include at least one of electroencephalogram (EEG), photo plethysmogram (PPG), galvanic skin reflex (GSR), respiration, and electromyogram (EMG).

In an embodiment, the state determining unit may determine a user state based on the measured bio signal while a first cognitive behavioral therapy image is being played, and the user state may include at least one of a cognitive state, an emotional state, and a craving state.

In an embodiment, the cognitive behavioral therapy image determining unit may include a cognitive behavior evaluating unit configured to determine a type of stimulation and a level of stimulation to provide to the user based on the determined user state, and a second cognitive behavioral therapy image determining unit configured to determine a second cognitive behavioral therapy image having the determined type and level of stimulation.

In an embodiment, the state determining unit may determine a second user state based on the measured bio signal while the second cognitive behavioral therapy image is being played, and the cognitive behavioral therapy image determining unit may determine a third cognitive behavioral therapy image based on the second user state.

In an embodiment, the second cognitive behavioral therapy image may be an image that is played after the first cognitive behavioral therapy image, or an image that is displayed together on a part of the first cognitive behavioral therapy image.

In an embodiment, the image providing unit may include at least one of a flat display, a curved display, and a head-mounted display.

A method for customized addiction therapy based on a bio signal according to an embodiment of the present disclosure includes measuring a bio signal from a user using a sensor, determining a user state based on the measured bio signal, determining a cognitive behavioral therapy image based on the determined user state, and providing the determined cognitive behavioral therapy image to the user.

In an embodiment, the bio signal may include at least one of EEG, PPG, GSR, respiration, and EMG.

In an embodiment, the determining of a user state may include determining a user state based on the measured bio signal while a first cognitive behavioral therapy image is being played, and the user state may include at least one of a cognitive state, an emotional state, and a craving state.

In an embodiment, the determining of a cognitive behavioral therapy image may include determining a type of stimulation and a level of stimulation to provide to the user based on the determined user state, and determining a second cognitive behavioral therapy image having the determined type and level of stimulation.

In an embodiment, the method for customized addiction therapy based on a bio signal may further include determining a second user state based on the measured bio signal while the second cognitive behavioral therapy image is being played, and determining a third cognitive behavioral therapy image based on the second user state.

In an embodiment, the second cognitive behavioral therapy image may be an image that is played after the first cognitive behavioral therapy image, or an image that is displayed together on a part of the first cognitive behavioral therapy image.

In an embodiment, the providing of the cognitive behavioral therapy image to the user may include providing the cognitive behavioral therapy image in a virtual reality environment using at least one of a flat display, a curved display, and a head-mounted display.

A computer-readable recording medium according to an embodiment of the present disclosure may have instructions for executing the method stored thereon.

The present disclosure can objectively identify a state of a subject by objectively monitoring the level of cognition (attention, execution, etc.), emotion (anger, depression, etc.), and craving of the subject through bio signals while the subject is doing realistic cognition and behavior in a virtual reality scenario. Furthermore, a fixed scenario is difficult to reflect the state of the user due to performing simple repeated cognitive behavioral therapy, while scenarios change depending on the state of the subject, increasing the effect of therapy through repeated cognitive behavioral therapy in realistic manner.

The effects of the present disclosure are not limited thereto, and other effects not mentioned herein will be clearly understood by those skilled in the art from the appended claims.

### BRIEF DESCRIPTION OF THE DRAWINGS

FIG. 1 is a block diagram of a system 1000 for customized addiction therapy based on a bio signal according to an embodiment of the present disclosure.

FIG. 2 shows a relationship between a bio signal 10 measured by a sensor 100 and a user state 20.

FIG. 3 is a block diagram of a cognitive behavioral therapy image determining unit 300 according to an embodiment of the present disclosure.

FIG. 4 is a diagram for illustrating the step for determining a second cognitive behavioral therapy image in an embodiment.

FIG. 5 shows a second cognitive behavioral therapy image provided according to another embodiment of the present disclosure.

FIG. 6 is a flowchart of a method for customized addiction therapy based on a bio signal according to an embodiment of the present disclosure.

FIG. 7 shows an exemplary cognitive behavioral therapy image for illustrating an embodiment of the present disclosure.

### DETAILED DESCRIPTION

The terminology used herein is for the purpose of describing particular embodiments only and is not intended to be limiting of the present disclosure. Unless otherwise expressly provided herein or the context otherwise requires, the singular forms are intended to include the plural forms as well. It will be further understood that the terms “comprises” and/or “comprising”, or “includes” and/or “including” as used herein specify the presence of stated features, integers, steps, operations, elements, components, or groups thereof, but do not preclude the presence or addition of one or more other features, integers, steps, operations, elements, components, or groups thereof.

Unless otherwise defined herein, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein. In the drawings, like reference numerals denote like elements. However, in describing the embodiments, when a certain detailed description of related known functions or elements is deemed as unnecessarily rendering the essence of the disclosure ambiguous, their detailed description is omitted herein. In addition, the size of each element in the drawings may be exaggerated for clarity, and does not represent the actually used size.

The embodiments described herein may take the form of entirely hardware, partially hardware and partially software, or entirely software. The term “unit”, “module”, “device”, or “system” as used herein indicates a computer-related entity such as hardware, a combination of hardware and software, or software. For example, a unit, module, device or system used herein may include, but is not limited to, a process being executed, a processor, an object, an executable, a thread of execution, a program, and/or a computer. For example, both a computer and an application running on the computer may correspond to the term unit, module, device, or system used herein.

The embodiments have been described with reference to the flowchart presented in the drawings. For the sake of simplicity, although the method has been illustrated and described as a series of blocks, the present disclosure is not limited to the order of the blocks, and some of the blocks may be placed with the other blocks in a different order from an order illustrated and described herein or may be concurrent with the other blocks, and a variety of other diverges, flow paths, and block sequences that achieve same or similar results may also be implemented. Further, the method according to an embodiment of the present disclosure may be implemented in the form of a computer program for performing a series of processes, and the computer program may also be recorded on a computer-readable recording medium.

The term “images” described herein may include scenarios having a predetermined story. Further the term images may include static image, video, or sound.

Hereinafter, the elements and features of the present disclosure are described using embodiments, but these embodiments are provided for illustration only, but are not intended to limit the present disclosure.

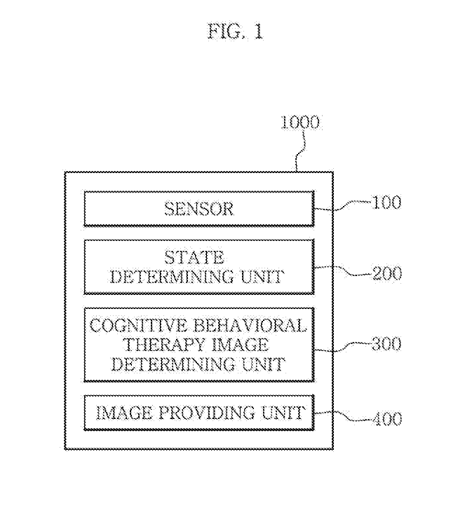

FIG. 1 is a block diagram of a system 1000 for customized addiction therapy based on a bio signal according to an embodiment of the present disclosure. Referring to FIG. 1, the system 1000 for customized addiction therapy based on a bio signal includes a sensor 100, a state determining unit 200, a cognitive behavioral therapy image determining unit 300 and an image providing unit 400.

The sensor 100 may measure a bio signal from a user. The sensor 100 may include at least one of an electroencephalography (EEG) sensor, a photo plethysmography (PPG) sensor, a galvanic skin reflex (GSR) sensor, a respiration sensor, and an electromyography (EMG) sensor. Accordingly, the bio signal may include at least one of EEG, PPG, GSR, respiration, and EMG. Referring to this, the sensor 100 according to an embodiment of the present disclosure may measure the bio signal from the central or autonomic nervous system of the user. The present disclosure is characterized by using the bio signal from the central (EEG) or autonomic nervous system (GSR, PPG, and respiration) as described above.

Furthermore, in an embodiment, the GSR or EMG sensor attached to the user to measure the bio signal of the user may be attached to any body part of the user.

The state determining unit 200 may determine a user state based on the bio signal measured by the sensor 100. The user state may include a type of state sorted by conventional classification that clinical or counseling psychologists have used to identify users' psychological state. For example, the user state may include at least one of a cognitive state, an emotional state and a craving state. Here, the cognitive state may include a state of attention and execution, the emotional state may include a state of anger, depression and sadness, and the craving state may include craving.

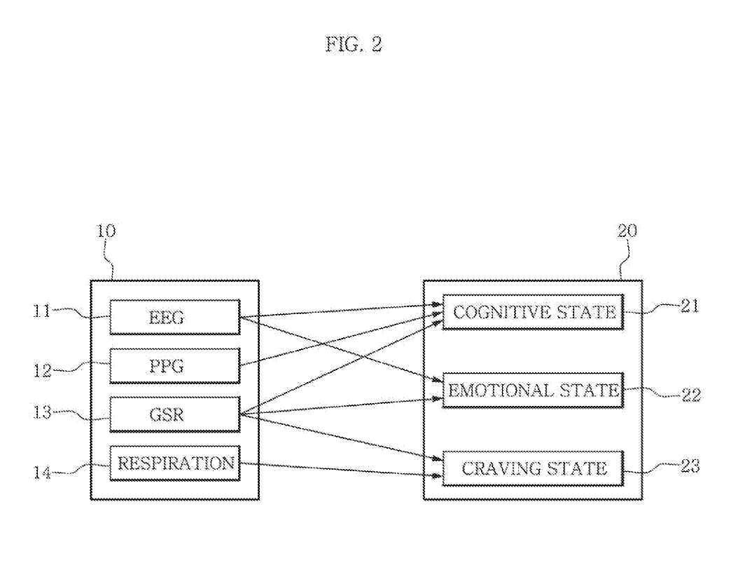

FIG. 2 shows a relationship between a bio signal 10 measured by the sensor 100 and a user state 20. The state determining unit 200 may determine a user state 21-23 based on at least one bio signal 11-14 measured by the sensor 100.

Referring to FIG. 2, in an example, the state determining unit 200 may determine a cognitive state 21 of a user by combining an EEG signal 11 and a PPG signal 12. A connection relationship (arrow) between the bio signal 11-14 and the user state 21-23 as expressed in FIG. 2 is for illustration only, and a bio signal not shown may be further used to determine a particular user state. For example, besides GSR and respiration signals, the state determining unit 200 may further use an EEG signal to determine a craving state.

In an embodiment, the state determining unit 200 may determine a user state based on the measured bio signal while a first cognitive behavioral therapy image is being played. The first cognitive behavioral therapy image may be any image provided to analyze the psychology of the user. For example, to treat a PC game addicted user, the first cognitive behavioral therapy image may be “a situation in which a PC is found in the room”, “a situation in which a PC is powered on in the room and is running a game”, “people are playing games in a PC gaming center”, etc. Here, the cognitive behavioral therapy image may include a static image or a dynamic image, and may also include audio information.

In the above example, when the sensor measures a bio signal of the user in “a situation in which a PC is found in the room”, the state determining unit 200 may determine a craving state based on the measured bio signal. In an example, the state determining unit 200 may calculate a value of the user state. The state determining unit 200 may calculate the level of state numerically, for example, craving has a value of 2 or anger has a value of 6.

Furthermore, in an example, a user state of at least one of cognition, emotion and craving may be determined based on the measured bio signal while the user is viewing the first cognitive behavioral therapy image. For example, the results of a cognitive state being 3 and craving being 5 may be derived.

In an embodiment, the state determining unit 200 may provide a clinical or counseling psychologist with the level of cognition (attention, execution, etc.), emotion (anger, depression, etc.), and craving through a particular value. Thus, the clinical or counseling psychologist may provide a therapy program necessary for a current user state. The therapy program may include an image to be subsequently played or behavior instructions.

In an embodiment of the present disclosure, the cognitive behavioral therapy image determining unit 300 and the image providing unit 400 may provide the user with the therapy program (a next image or giving the user behavior instructions, a warning, etc.).

In an embodiment, the cognitive behavioral therapy image determining unit 300 may determine a cognitive behavioral therapy image based on the determined user state. In another embodiment, the cognitive behavioral therapy image determining unit 300 may determine a cognitive behavioral therapy image to be subsequently played based on the determined user state and stimulation information of the played cognitive behavioral therapy image.

To this end, the system 1000 for customized addiction therapy based on a bio signal may further include a database (not shown) to store cognitive behavioral therapy images and stimulation information associated with each cognitive behavioral therapy image. The database may be included in the system 1000 for customized addiction therapy based on a bio signal, or may be stored in an external device. Furthermore, the stimulation information may include the type and level of stimulation.

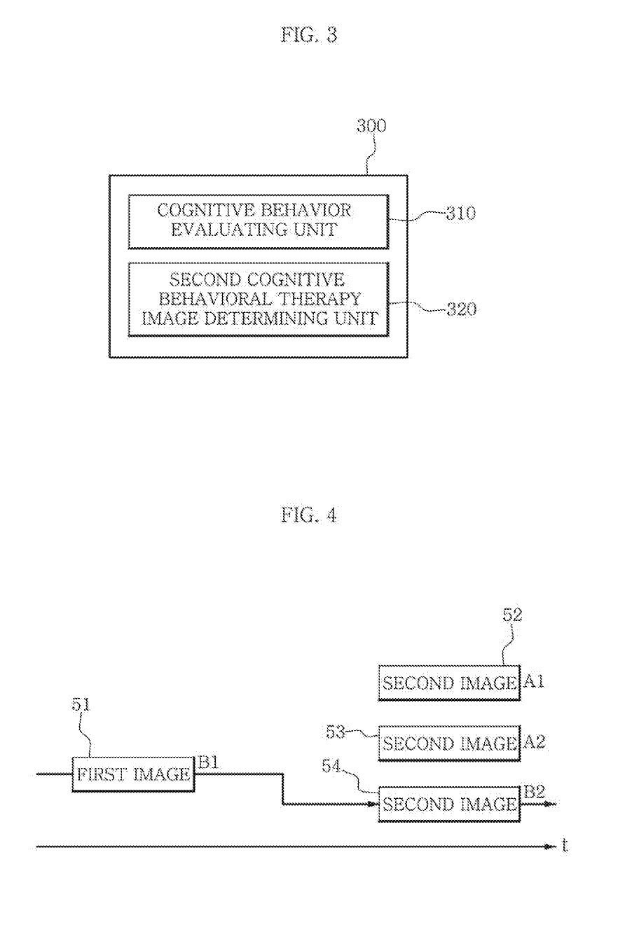

FIG. 3 is a block diagram of the cognitive behavioral therapy image determining unit 300 according to an embodiment of the present disclosure. Referring to FIG. 3, the cognitive behavioral therapy image determining unit 300 includes a cognitive behavior evaluating unit 310 to determine the type of stimulation and the level of stimulation to provide to the user based on the determined user state, and a second cognitive behavioral therapy image determining unit 320 to determine a second cognitive behavioral therapy image having the determined type and level of stimulation.

For example, when the user state obtained from the user viewing the first cognitive behavioral therapy image is craving 2, the cognitive behavior evaluating unit 310 may determine the type of stimulation to provide to the user to be craving and the level of stimulation to be 1 or 3. Here, the stimulation may be cognition, emotion or craving corresponding to the user state. When the level of craving is 1, lower stimulation is provided, and when the level of craving is 3, higher stimulation is provided.

FIG. 4 is a diagram for illustrating the step for determining a second cognitive behavioral therapy image in an embodiment. Referring to FIG. 4, the horizontal axis denotes the time t, and alphabets and numbers indicated at the right side of each image 51-54 denote the type and level of stimulation. For example, A1 indicates that the level of craving is 1, and B2 indicates that the level of anger is 2.

Referring to FIG. 4, stimulation information of a first cognitive behavioral therapy image 51 is B1. If it is assumed that a user state obtained from a user viewing the first cognitive behavioral therapy image 51 is B1, the cognitive behavioral therapy image determining unit 300 may determine a second cognitive behavioral therapy image based on the user state B1 and stimulation information B1 of the played first cognitive behavioral therapy image 51. Referring to FIG. 4, the cognitive behavioral therapy image determining unit 300 may determine a second cognitive behavioral therapy image 54 providing anger 2 B2 from the second cognitive behavioral therapy images 52-54. Thus, after viewing the first cognitive behavioral therapy image 51, the user may view the second cognitive behavioral therapy image 54 having a higher level of anger.

In an embodiment, the state determining unit 200 may determine a second user state based on the measured bio signal while the second cognitive behavioral therapy image is being played. Subsequently, the cognitive behavioral therapy image determining unit 300 may determine a third cognitive behavioral therapy image based on the second user state. That is, according to an embodiment of the present disclosure, an optimal image may be determined based on continuous image playing and biometric information obtained from each image and may be provided to the user.

In an embodiment, the second cognitive behavioral therapy image may be an image that is played after the first cognitive behavioral therapy image, or an image that is displayed together on a part of the first cognitive behavioral therapy image. This relationship may be equally applied to a relationship between the second cognitive behavioral therapy image and the third cognitive behavioral therapy image.

Referring to FIG. 4, after the first cognitive behavioral therapy image is played, the second cognitive behavioral therapy image is played. Because the first and second cognitive behavioral therapy images have different types and levels of stimulation, the user may have cognitive behavioral therapy with different experiences and stimulations in each image.

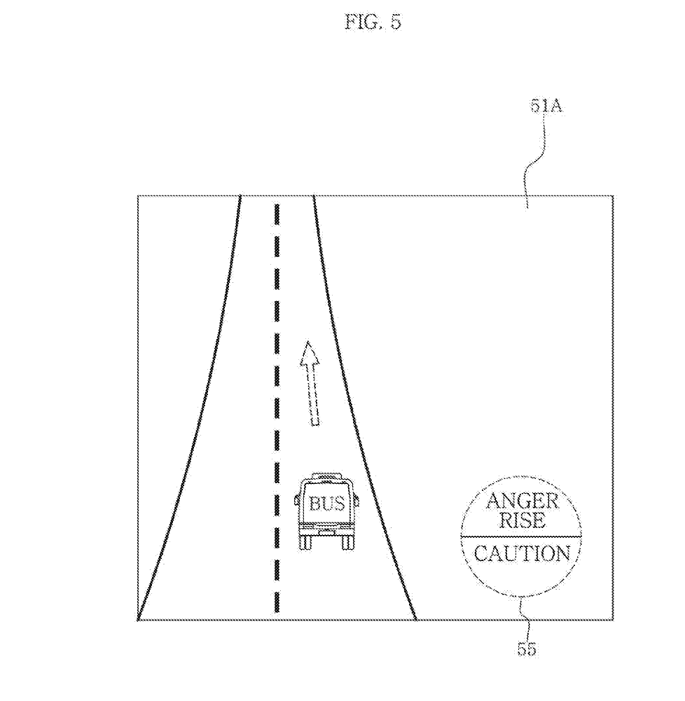

FIG. 5 shows a second cognitive behavioral therapy image provided according to another embodiment of the present disclosure. Although the horizontal axis denotes the time in FIG. 4, FIG. 5 shows a scene of a first cognitive behavioral therapy image 51A displayed on a display. Referring to FIG. 5, a second cognitive behavioral therapy image 55 is displayed in an overlapping manner with a part of a first cognitive behavioral therapy image 51A.

FIG. 5 is an image showing a situation of missing a bus, and stimulation information of anger 3 is assumed. When a user state obtained from a measured bio signal is determined to exceed a preset level of anger while a user is viewing the first cognitive behavioral therapy image 51A, the cognitive behavioral therapy image determining unit 300 may provide the second cognitive behavioral therapy image 55 as a warning message to the user as shown in FIG. 5. Thus, the user may recognize that his/her current state has excessive anger.

The image providing unit 400 may provide the user with the determined cognitive behavioral therapy image. That is, the image providing unit 400 may provide the user with the first to third cognitive behavioral therapy images.

Furthermore, in an embodiment, the image providing unit 400 may include at least one of a flat display, a curved display, and a head-mounted display (HMD). To increase the immersion of the user, the image providing unit 400 may provide the cognitive behavioral therapy image in a virtual reality environment through a head-mounted display. For example, a situation in which the user waits for a bus at the bus stop and misses a bus may be provided through a virtual reality environment.

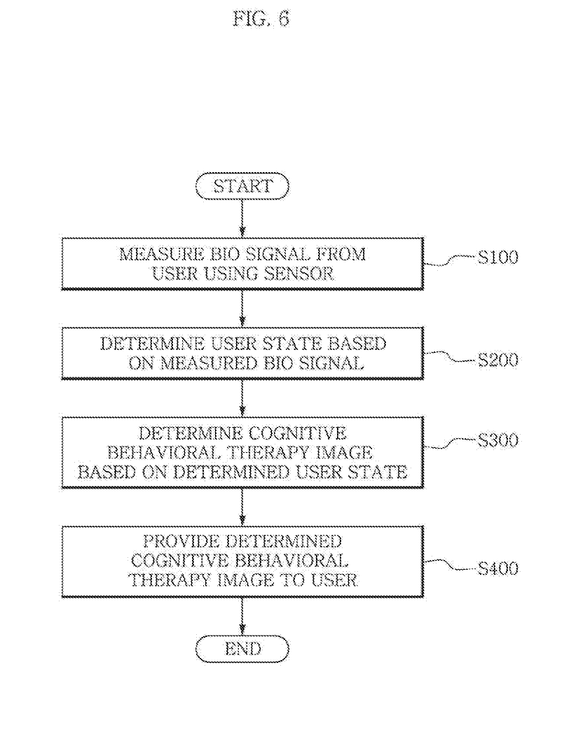

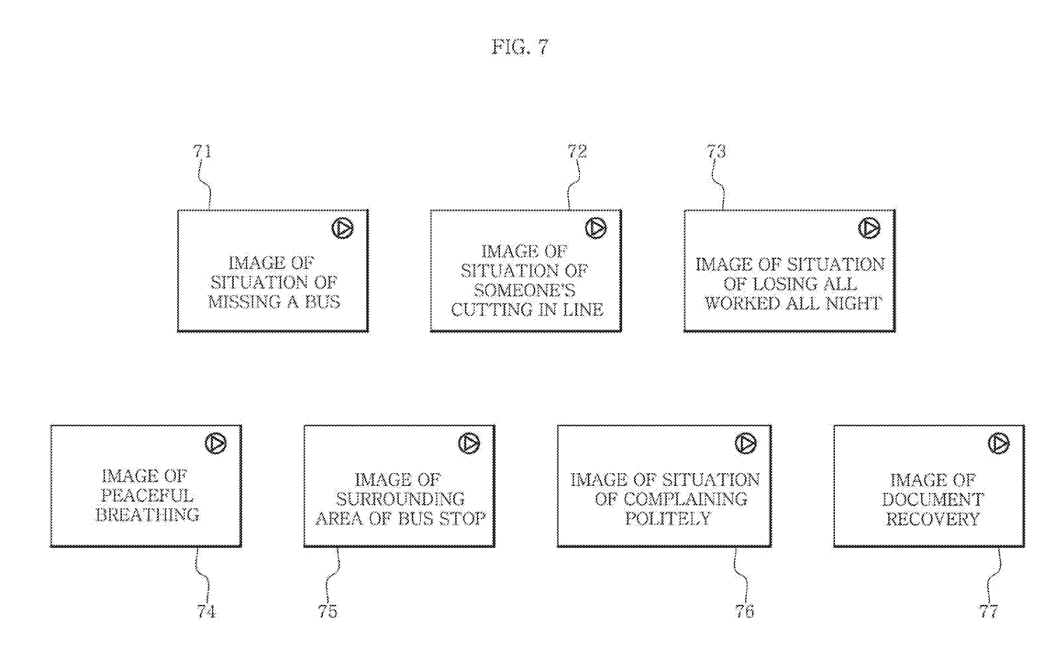

FIG. 6 is a flowchart of a method for customized addiction therapy based on a bio signal according to an embodiment of the present disclosure. Referring to FIG. 6, the method for customized addiction therapy based on a bio signal includes measuring a bio signal from a user using a sensor (S100), determining a user state based on the measured bio signal (S200), determining a cognitive behavioral therapy image based on the determined user state (S300) and providing the determined cognitive behavioral therapy image to the user (S400). Here, the bio signal includes, but is not limited to, at least one of EEG, PPG, GSR, respiration, and EMG. Furthermore, the user state may include at least one of a cognitive state, an emotional state and a craving state.

The determining of a user state (S200) may include determining a user state based on the measured bio signal while a first cognitive behavioral therapy image is being played.

The determining of a cognitive behavioral therapy image (S300) may include determining the type of stimulation and the level of stimulation to provide to the user based on the determined user state, and determining a second cognitive behavioral therapy image having the determined type and level of stimulation.

In another embodiment, the method for customized addiction therapy based on a bio signal may further include determining a second user state based on the measured bio signal while the second cognitive behavioral therapy image is being played, and determining a third cognitive behavioral therapy image based on the second user state.

Here, the second cognitive behavioral therapy image may be an image that is played after the first cognitive behavioral therapy image, or an image that is displayed together on a part of the first cognitive behavioral therapy image.

In an embodiment, the providing of the cognitive behavioral therapy image to the user (S400) may include providing the cognitive behavioral therapy image in a virtual reality environment using at least one of a flat display, a curved display, and a head-mounted display.

A computer-readable recording medium according to an embodiment of the present disclosure may store instructions for executing the method for customized addiction therapy based on a bio signal.

Furthermore, a program according to another embodiment includes instructions for executing the method for customized addiction therapy based on a bio signal, and may be stored in the computer-readable recording medium.

Hereinafter, provided is a description of the case in which the system for customized addiction therapy based on a bio signal according to an embodiment of the present disclosure is applied to cognitive behavioral therapy for anger control.

### Embodiment

FIG. 7 shows an exemplary cognitive behavioral therapy image for illustrating an embodiment of the present disclosure. Referring to FIG. 7, the system 1000 for customized addiction therapy based on a bio signal provides a user with a situation in which the user waits for a bus at the bus stop, is late for an appointment, and misses a bus due to someone, through an image 71. In this instance, when the level of anger as a user state obtained from a bio signal of the user exceeds a predetermined value, the system 1000 for customized addiction therapy based on a bio signal may help the user breathe peacefully through an image 74. As a result, the user may take his/her time to control anger. Subsequently, the system 1000 for customized addiction therapy based on a bio signal provides the user with a situation in which the user waits for a bus once again, through an image 75. The user is monitored to determine if the user expresses anger in the same way as before in the situation in which the user waits for a bus through the image 75.

Furthermore, in another embodiment, in a situation in which the user misses a bus late for an appointment as provided through an image 71, when the user does not get angry and maintains the peace, the system 1000 for customized addiction therapy based on a bio signal provides an image 72 providing greater anger. When a situation in which someone cuts in the line is provided, determination is made as to whether the user gets angry or maintains the peace in response to this level of stimulation. If the level of anger exceeds a predetermined value in the situation provided by the image 72, the system 1000 for customized addiction therapy based on a bio signal may give the user an amount of time to control anger through the image 74. Subsequently, behavior guidelines may be provided through an image 76 to allow the user to control anger and express the controlled anger.

While the present disclosure has been hereinabove described by particular matters such as specific elements and a limited number of embodiments and drawings, this is only provided to assist a full and complete understanding of the present disclosure, and the present disclosure is not limited to the above embodiments and various modifications and variations can be conceived by those skilled in the art from the disclosure.

Therefore, the spirit of the present disclosure shall not be confined to the disclosed embodiments, and the appended claims and equal or equivalent variations thereto will fall within the spirit and scope of the present disclosure.

What is claimed is:

1. A system for customized addiction therapy based on a bio signal, comprising:
a sensor configured to measure a bio signal from a user;
a state determining unit configured to determine a user addicted state based on the measured bio signal;
a cognitive behavioral therapy image determining unit configured to determine a cognitive behavioral therapy image based on the determined user state; and
an image providing unit configured to provide the determined cognitive behavioral therapy image to the user.

2. The system for customized addiction therapy based on a bio signal according to claim 1, wherein the bio signal includes at least one of electroencephalogram (EEG), photo plethysmogram (PPG), galvanic skin reflex (GSR), respiration, and electromyogram (EMG).

3. The system for customized addiction therapy based on a bio signal according to claim 1, wherein the state determining unit determines a user addicted state based on the measured bio signal while a first cognitive behavioral therapy image is being played, and
the user state includes at least one of a cognitive state, an emotional state, and a craving state.

4. The system for customized addiction therapy based on a bio signal according to claim 3, wherein the cognitive behavioral therapy image determining unit comprises:
a cognitive behavior evaluating unit configured to determine a type of stimulation and a level of stimulation to provide to the user based on the determined user state; and
a second cognitive behavioral therapy image determining unit configured to determine a second cognitive behavioral therapy image having the determined type and level of stimulation.

5. The system for customized addiction therapy based on a bio signal according to claim 4, wherein the state determining unit determines a second user state based on the measured bio signal while the second cognitive behavioral therapy image is being played, and
the cognitive behavioral therapy image determining unit determines a third cognitive behavioral therapy image based on the second user state.

6. The system for customized addiction therapy based on a bio signal according to claim 4, wherein the second cognitive behavioral therapy image is an image that is played after the first cognitive behavioral therapy image, or an image that is displayed together on a part of the first cognitive behavioral therapy image.

7. The system for customized addiction therapy based on a bio signal according to claim 1, wherein the image providing unit includes at least one of a flat display, a curved display, and a head-mounted display.

8. A method for customized addiction therapy based on a bio signal, comprising:
measuring a bio signal from a user using a sensor;
determining a user state based on the measured bio signal;
determining a cognitive behavioral therapy image based on the determined user state; and
providing the determined cognitive behavioral therapy image to the user.

9. The method for customized addiction therapy based on a bio signal according to claim 8, wherein the bio signal includes at least one of electroencephalogram (EEG), photo plethysmogram (PPG), galvanic skin reflex (GSR), respiration, and electromyogram (EMG).

10. The method for customized addiction therapy based on a bio signal according to claim 8, wherein the determining of a user state comprises determining a user state based on the measured bio signal while a first cognitive behavioral therapy image is being played, and
the user state includes at least one of a cognitive state, an emotional state, and a craving state.

11. The method for customized addiction therapy based on a bio signal according to claim 10, wherein the determining of a cognitive behavioral therapy image comprises:
determining a type of stimulation and a level of stimulation to provide to the user based on the determined user state; and
determining a second cognitive behavioral therapy image having the determined type and level of stimulation.

12. The method for customized addiction therapy based on a bio signal according to claim 11, further comprising:
determining a second user state based on the measured bio signal while the second cognitive behavioral therapy image is being played; and
determining a third cognitive behavioral therapy image based on the second user state.

13. The method for customized addiction therapy based on a bio signal according to claim 11, wherein the second cognitive behavioral therapy image is an image that is played after the first cognitive behavioral therapy image, or an image that is displayed together on a part of the first cognitive behavioral therapy image.

14. The method for customized addiction therapy based on a bio signal according to claim 8, wherein the providing of the cognitive behavioral therapy image to the user comprises providing the cognitive behavioral therapy image in a virtual reality environment using at least one of a flat display, a curved display, and a head-mounted display.

15. A computer-readable recording medium having stored thereon instructions for executing the method according to claim 8.
